(19) United States
(12) Patent Application Publication
(10) Pub. No.: US 2024/0404547 A1
(43) Pub. Date: Dec. 5, 2024

(54) SOUND SOURCE DETERMINING METHOD AND SYSTEM, ELECTRONIC DEVICE AND READABLE STORAGE MEDIUM

(71) Applicant: LUXSHARE PRECISION INDUSTRY COMPANY LIMITED, Shenzhen (CN)

(72) Inventor: Guanhsiung WANG, Shenzhen (CN)

(73) Assignee: LUXSHARE PRECISION INDUSTRY COMPANY LIMITED, Shenzhen (CN)

(21) Appl. No.: 18/678,936

(22) Filed: May 30, 2024

(30) Foreign Application Priority Data
May 30, 2023 (CN) 202310631553.6

(51) Int. Cl. G10L 25/51; G06T 7/70; G10L 25/09; H04R 1/40; H04R 3/00

(52) U.S. Cl. CPC G10L 25/51; G06T 7/70; G10L 25/09; H04R 1/406; H04R 3/005; G06T 2207/10048; G06T 2207/30196

(57) ABSTRACT

A sound source determining method and system and an electronic device are disclosed. The sound source determining method includes: obtaining initial audio information collected in real time; performing audio recognition processing on the initial audio information to obtain an audio recognition result; using the initial audio information corresponding to the audio recognition result as target audio information in a case that the audio recognition result indicates that the initial audio information meets a preset audio recognition condition; performing audio information activity detection on the target audio information to obtain target audio activity information; and performing sound source positioning on a sound producing object corresponding to the target audio activity information according to sound source positioning parameters corresponding to the target audio activity information to obtain target position information of the sound producing object.

### CROSS-REFERENCE TO RELATED APPLICATIONS

The present disclosure claims all benefits of the Chinese patent application with the application number 202310631553.6 and entitled “Sound Source Determining Method and System, Electronic Device and Readable Storage Medium” filed to China National Intellectual Property Administration on May 30, 2023, which is incorporated herein by reference in its entirety.

### FIELD

The present disclosure generally relates to the field of sound source positioning, more particularly, to a sound source determining method and system, an electronic device and a readable storage medium.

### BACKGROUND

Through a sound recognition technology, human voices, snoring, abnormal noises, and the sound of moving objects during movement can be recognized, and therefore, it can be widely applied in various aspects such as speech processing and fault detection. In the related art, the sound recognition technology only targets a single object and has high requirements for the sound production quality of an object to be recognized, resulting in the sound recognition technology only being able to adapt to situations with a low environmental change degree, thus reducing the universality and stability of the sound recognition technology.

### SUMMARY

In one aspect, the present disclosure relates to a sound source determining method, including: obtaining initial audio information collected in real time; performing audio recognition processing on the initial audio information to obtain an audio recognition result; using the initial audio information corresponding to the audio recognition result as target audio information in a case that the audio recognition result indicates that the initial audio information meets a preset audio recognition condition; performing audio information activity detection on the target audio information to obtain target audio activity information; and performing sound source positioning on a sound producing object corresponding to the target audio activity information according to sound source positioning parameters corresponding to the target audio activity information to obtain target position information of the sound producing object.

In some implementations, obtaining the initial audio information collected in real time includes: arranging sound receiving assemblies in at least two different azimuths, and performing audio information collection in real time based on the sound receiving assemblies in the at least two different azimuths to obtain the initial audio information.

In some implementations, the preset audio recognition condition includes a preset audio signal frequency range and a preset audio signal sound pressure range, and performing audio recognition processing on the initial audio information to obtain the audio recognition result includes: performing frequency feature recognition or sound pressure feature recognition on the initial audio information to obtain the audio recognition result; and using the initial audio information corresponding to the audio recognition result as the target audio information in a case that the audio recognition result indicates that the initial audio information meets the preset audio recognition condition includes: using the initial audio information corresponding to the audio recognition result as the target audio information if the audio recognition result indicates that a frequency of the initial audio information meets the preset audio signal frequency range and a sound pressure of the initial audio information meets the preset audio signal sound pressure range.

In some implementations, performing audio information activity detection on the target audio information to obtain the target audio activity information includes: obtaining an audio signal amplitude and a zero crossing rate corresponding to the target audio information, the zero crossing rate being a number of times sampling information corresponding to the target audio information crosses a zero point, and the sampling information being obtained after performing many times of sampling on the target audio information; comparing the audio signal amplitude with a preset amplitude threshold to obtain an amplitude threshold comparison result; comparing the zero crossing rate with a preset zero crossing rate threshold to obtain a zero crossing rate comparison result; and determining the target audio information as the target audio activity information in a case that the amplitude threshold comparison result indicates that the audio signal amplitude is greater than the amplitude threshold and the zero crossing rate comparison result indicates that the zero crossing rate is less than or equal to the zero crossing rate threshold.

In some implementations, performing audio information activity detection on the target audio information to obtain the target audio activity information includes: obtaining an audio signal amplitude or a zero crossing rate corresponding to the target audio information, the zero crossing rate being a number of times sampling information corresponding to the target audio information crosses a zero point, and the sampling information being obtained after performing many times of sampling on the target audio information; comparing the audio signal amplitude with a preset amplitude threshold to obtain an amplitude threshold comparison result; or comparing the zero crossing rate with a preset zero crossing rate threshold to obtain a zero crossing rate comparison result; and determining the target audio information as the target audio activity information in a case that the amplitude threshold comparison result indicates that the audio signal amplitude is greater than the amplitude threshold or the zero crossing rate comparison result indicates that the zero crossing rate is less than or equal to the zero crossing rate threshold.

In some implementations, the sound source positioning parameters include a time difference of the respective sound receiving assemblies receiving audio signals, a spacing distance between the respective sound receiving assemblies, an audio signal propagation velocity and a sampling rate of the target audio information, and performing sound source positioning on the sound producing object corresponding to the target audio activity information according to the sound source positioning parameters corresponding to the target audio activity information to obtain the target position information of the sound producing object includes: performing angle positioning on the sound producing object based on the time difference of the respective sound receiving assemblies receiving the audio signals, the spacing distance between the respective sound receiving assemblies, the audio signal propagation velocity and the sampling rate of the target audio information, to obtain azimuth angle parameters between the sound producing object and the respective sound receiving assemblies; performing distance positioning estimation on the sound producing object based on the audio signal propagation velocity and a time difference of the respective sound receiving assemblies receiving audio signals in front and back cycles, to obtain linear distance parameters between the sound producing object and the sound receiving assemblies; and determining relative position information between the sound producing object and the sound receiving assemblies in a three-dimensional space according to the azimuth angle parameters and the linear distance parameters, and using the relative position information as the target position information.

In some implementations, an image obtaining region corresponding to an infrared image obtaining module is adjusted to obtain a target image obtaining region, and the target image obtaining region includes the sound producing object.

In some implementations, an infrared image shooting operation is executed on the target image obtaining region through the infrared image obtaining module to obtain infrared image attitude information of the sound producing object, and the infrared image attitude information is stored, the infrared image attitude information being used for obtaining a corresponding attitude correction method.

In another aspect, the present disclosure relates to a sound source determining system, including a sound receiving module, configured to obtain initial audio information collected in real time; a processing module, configured to perform audio recognition processing on the initial audio information to obtain an audio recognition result; and use the initial audio information corresponding to the audio recognition result as target audio information in a case that the audio recognition result indicates that the initial audio information meets a preset audio recognition condition; a detecting module, configured to perform audio information activity detection on the target audio information to obtain target audio activity information; and a positioning module, configured to perform sound source positioning on a sound producing object corresponding to the target audio activity information according to sound source positioning parameters corresponding to the target audio activity information to obtain target position information of the sound producing object.

In yet another aspect, the present disclosure relates to an electronic device, including one or more processors; and a storage apparatus, configured to store one or more computer programs, wherein the one or more computer programs, when executed by the one or more processors, cause the electronic device to implement the sound source determining method of the present disclosure.

In yet another aspect, the present disclosure relates to a computer-readable storage medium, storing a computer program thereon, wherein the computer program, when executed by a processor of an electronic device, causes the electronic device to execute the sound source determining method of the present disclosure.

In another aspect, the present disclosure relates to a computer program product, including a computer program, the computer program being stored in a computer-readable storage medium. A processor of an electronic device reads the computer program from the computer-readable storage medium, and the processor executes the computer program to cause the electronic device to execute the sound source determining method of the present disclosure.

### BRIEF DESCRIPTION OF DRAWINGS

The accompanying drawings herein are incorporated into the specification and constitute a part of the specification, show embodiments consistent with the present disclosure, and together with the specification, are used to explain the principle of the present disclosure. Apparently, the accompanying drawings in the following description are only some embodiments of the present disclosure, and for those of ordinary skill in the art, on the premise of no creative labor, other accompanying drawings can further be obtained from these accompanying drawings. In the accompanying drawings:

FIG. 1 shows a basic flow diagram of a sound source determining method illustrated by an exemplary embodiment of the present disclosure;

FIG. 2 shows a basic structural diagram of a sound source determining system illustrated by an exemplary embodiment of the present disclosure;

FIG. 3 shows a basic structural diagram of another sound source determining system illustrated by an exemplary embodiment of the present disclosure;

FIG. 4 shows a basic flow diagram of a sound source determining method illustrated by an exemplary embodiment of the present disclosure; and

FIG. 5 shows a schematic structural diagram of a computer system of an electronic device suitable for implementing one embodiment of the present disclosure.

### DETAILED DESCRIPTION

Exemplary embodiments will be described in detail here, and examples of the exemplary embodiments are shown in the accompanying drawings. When the following description refers to the accompanying drawings, unless otherwise indicated, the same numbers in different accompanying drawings indicate the same or similar elements. The implementations described in the following exemplary embodiments do not represent all implementations consistent with the present disclosure. Rather, they are merely examples of apparatuses and methods consistent with some aspects of the present disclosure as detailed in the appended claims.

The block diagram shown in the accompanying drawings is only a functional entity and does not necessarily correspond to physically independent entities. That is, these functional entities may be implemented in a software form, or in one or more hardware modules or integrated circuits, or in different networks and/or processor apparatuses and/or microcontroller apparatuses.

The flow diagram shown in the accompanying drawings is only exemplary description, does not necessarily include all contents and operations, and is not necessarily executed in the described order either. For example, some operations may also be divided, while others may be merged or partially merged, so the actual order of execution may change according to the actual situation.

It should also be noted that: “a plurality of” referred to in the present disclosure refers to two or more. “And/or” describes the association relationship of associated objects, which means that there may be three kinds of relationships, for example, A and/or B may represent that there are three kinds of situations: A alone, A and B at the same time, and B alone. The character “/” universally indicates that associated objects are in an “or” relationship.

### Embodiment 1

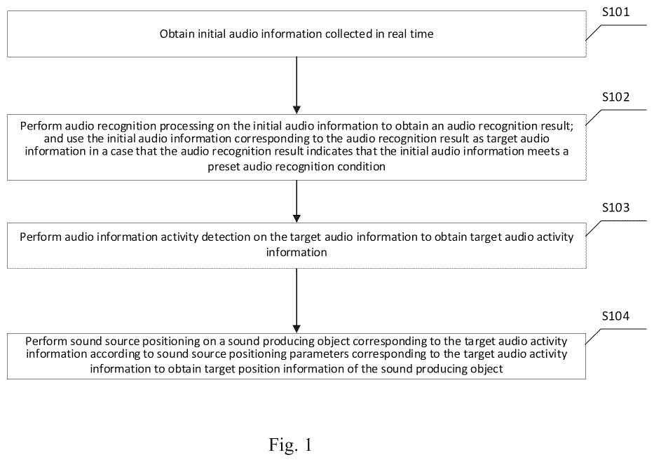

The present embodiment provides a sound source determining method, and as shown in FIG. 1, the method includes:
S101, obtaining initial audio information collected in real time;
S102, performing audio recognition processing on the initial audio information to obtain an audio recognition result; and using the initial audio information corresponding to the audio recognition result as target audio information in a case that the audio recognition result indicates that the initial audio information meets a preset audio recognition condition;
S103, performing audio information activity detection on the target audio information to obtain target audio activity information; and
S104, performing sound source positioning on a sound producing object corresponding to the target audio activity information according to sound source positioning parameters corresponding to the target audio activity information to obtain target position information of the sound producing object.

It can be understood that, the above initial audio information is digital information obtained by conversion based on a sound collected in real time, wherein the sound collected in real time may be a sound produced by one sound producing object or sounds produced by a plurality of sound producing objects; if it is determined that the received sound is produced by one sound producing object, this sound is directly converted to the digital information to obtain the initial audio information; and if it is determined that the received sounds are produced by the plurality of sound producing objects, the collected sounds need to be separated, and initial audio information corresponding to each sound producing object is obtained based on the separated sounds. A method of separating speech characters for sounds is not limited in this example, and relevant personnel may flexibly select a speech character separation method to perform speech character separation on the sounds.

In some implementations, after the sound receiving assemblies receive a sound, an analog signal of the sound is obtained, the analog signal is vulnerable to the interference of the surroundings due to its variability and is not conducive to subsequent signal reproduction and signal processing, and thus the analog signal needs to be converted to a corresponding digital signal to obtain the corresponding initial audio information.

A manner of converting the analog signal to the digital signal is not limited in the present embodiment, and description is made below by taking converting the analog signal to the digital signal to obtain the initial audio information as an example: converting the analog signal to the digital signal includes three main steps of sampling, quantification and coding.

Sampling: the analog signal is digitized on a time axis with a certain sampling rate. In some implementations, first, a plurality of points (e.g., points on a wave corresponding to 1-10 in the figure) are taken in sequence along the time axis according to a fixed time interval T (assuming T=0.1 s). At the moment, T is called a sampling cycle, and a reciprocal of T is a target frequency (f=1/T=10 Hz) in this time of sampling, where f represents the number of times of sampling every second, with a unit being Hz. Obviously, the higher the target frequency, the more sampling points in unit time, and the better the representation of an original waveform (if countless points are collected densely at a high frequency, it is equivalent to that the original waveform is recorded completely). It can be understood that, the higher the target frequency, the more the sampling points, and the better the representation of the original waveform. Reference may be made to the Nyquist sampling theorem for more detailed description: the target frequency f must be greater than 2 times a maximum vibration frequency fmax of an original audio signal (i.e., f>2*fmax, fmax being called a Nyquist frequency) in order for a sampling result to be used for rebuilding the original audio signal completely; and if the sampling rate is lower than 2*fmax, then audio sampling has distortion. For example, for performing sampling on an original audio with the maximum frequency fmax=8 KHz, the target frequency f is at least 16 KHz.

It can be understood that, the specific sampling rate may be flexibly set by relevant personnel according to actual using requirements, which is not limited in the present embodiment.

Second step, quantification: the analog signal is digitized on an amplitude axis with certain precision. In some implementations, after sampling is completed, the second step, quantification, of audio digitization is performed next. Sampling is digitizing the audio signal on the time axis to obtain the plurality of sampling points; while quantification is performing digitization in an amplitude direction to obtain an amplitude value of each sampling point.

Third step, coding: sampled/quantified data are recorded according to a particular format. In some implementations, after quantification, the amplitude value of each sampling point is obtained. Next, the last step, coding, of digitization of the audio signal is carried out. Coding is to transform the amplitude quantified value of each sampling point into a binary byte sequence that can be understood by a computer.

Reference is made to a table at a coding part, a sample number is a sample sampling sequence, a sample value (decimal) is a quantified amplitude value. A sample value (binary) is coded data converted from the amplitude value. Finally, the binary byte sequence in the form of “0” and “1” is obtained, namely a discrete digital signal. What is obtained here is an uncompressed audio sampling data bare stream, which is also called pulse code modulation (PCM) audio data. In practical applications, other coding algorithms are also often used for further compression, which are not limited in the present embodiment.

In some implementations, the above collected sound may be a sound produced when the sound producing object breathes, the sound during breathing is collected and converted to a digital signal to obtain the initial audio information, in a case that the target audio recognition result indicates that the initial audio information meets the preset audio recognition condition, sound source positioning is performed on the sound producing object corresponding to the initial audio information according to the sound source positioning parameters corresponding to the initial audio information so as to obtain the target position information of the sound producing object, in a case that the target audio recognition result indicates that the initial audio information meets the preset audio recognition condition, it indicates that the sound producing object corresponding to the initial audio information is a particular sound producing object, sound source positioning is performed on the sound producing object directly according to the initial audio information, the position information of the particular sound producing object is obtained in a multi-person scenario quickly, accurately and stably, and the problem that the particular sound producing object cannot be positioned in the related art is avoided.

In some implementations, obtaining the initial audio information collected in real time includes:
arranging sound receiving assemblies in at least two different azimuths, and performing audio collection in real time based on the sound receiving assemblies in the at least two different azimuths to obtain the initial audio information.

In some implementations, the sound receiving assemblies include but are not limited to a microphone or a terminal device (such as a mobile phone and a smart watch) provided with a microphone.

Taking an example that the sound receiving assemblies are microphones below, the microphones are arranged in at least two different azimuths of one region respectively, and audio information collection is performed in real time through the at least two microphones synchronously, that is, when the audio information is collected, the at least two microphones work synchronously to make the collected audio information more complete, wherein the above two microphones are a left-channel microphone and a right-channel microphone arranged on a PCB.

It can be understood that, the quantity and arranging azimuths of the sound receiving assemblies are not limited in the present embodiment, as long as the sound receiving assemblies are arranged in at least two different azimuths of one region. In some implementations, there are two sound receiving assemblies, one placed on the left and the other on the right. In some implementations, there are three sound receiving assemblies, two placed on the left and the other on the right. In some implementations, there are four sound receiving assemblies, which are placed on the east, the south, the west and the north respectively.

In some implementations, the preset audio recognition condition includes a preset audio signal frequency range and a preset audio signal sound pressure range, and performing audio recognition processing on the initial audio information to obtain the target audio recognition result includes: performing frequency feature recognition or sound pressure feature recognition on the initial audio information to obtain the audio recognition result; and using the initial audio information corresponding to the audio recognition result as the target audio information in a case that the audio recognition result indicates that the initial audio information meets the preset audio recognition condition includes: using the initial audio information corresponding to the audio recognition result as the target audio information if the audio recognition result indicates that a frequency of the initial audio information meets the preset audio signal frequency range and a sound pressure of the initial audio information meets the preset audio signal sound pressure range.

In the step of performing frequency feature recognition or sound pressure feature recognition on the initial audio information to obtain the initial audio recognition result, the audio signal frequency or the audio signal sound pressure of the initial audio information is obtained first, then the frequency of the initial audio information matches with the preset audio signal frequency range and the sound pressure of the initial audio information matches with the preset audio signal sound pressure range, and if the frequency of the initial audio information is within the preset audio signal frequency range and the sound pressure of the initial audio information is within the preset audio signal sound pressure range, it is determined that the initial audio information corresponding to the audio recognition result is used as the target audio information in a case that the audio recognition result indicates that the initial audio information meets the preset audio recognition condition; otherwise, if the frequency of the initial audio information is not within the preset audio signal frequency range or the sound pressure of the initial audio information is not within the preset audio signal sound pressure range, it is determined that the initial audio information corresponding to the audio recognition result is not used as the target audio information in a case that the audio recognition result indicates that the initial audio information does not meet the preset audio recognition condition.

In some implementations, the initial audio information corresponding to the audio recognition result is used as the target audio information if the audio recognition result indicates that the frequency of the initial audio information meets the preset audio signal frequency range or the sound pressure of the initial audio information meets the preset audio signal sound pressure range.

It can be understood that, the preset audio signal frequency range and the preset audio signal sound pressure range are determined by relevant personnel according to actual using requirements. In some implementations, the relevant personnel want to recognize a sound producing object having a snoring situation, at the moment, a snore complex sound frequency is obtained, and then it is determined that the preset audio signal frequency range is met according to the snore complex sound frequency; and a decibel-sound pressure level during snoring of each area of the throat is obtained, and then it is determined that the preset audio signal sound pressure range is met according to the decibel-sound pressure level during snoring of each area of the throat.

In some implementations, the obtained decibel-sound pressure level during snoring of each area of the throat is as follows:
(1) an average decibel-sound pressure level (dBSPL) of a snore at the soft palate is 36.58 dBSPL;
(2) an average decibel-sound pressure level of a snore at the epiglottis is 23.49 dBSPL; and
(3) an average decibel-sound pressure level of a snore at the root of tongue is 16.3 dBSPL.

In some implementations, to avoid errors, an error amount is set to be 3 dBSPL, and then the obtained preset audio signal sound pressure range is 33.58 dBSPL to 39.58 dBSPL, 33.58 dBSPL to 39.58 dBSPL, and 13.3 dBSPL to 16.3 dBSPL; the initial audio information may be a snore complex sound, including a plurality of snore audios, and if a sound pressure in the initial audio information falls into the range of 33.58 dBSPL to 39.58 dBSPL, a sound pressure in the initial audio information falls into the range of 33.58 dBSPL to 39.58 dBSPL and a sound pressure of the initial audio information falls into the range of 13.3 dBSPL to 16.3 dBSPL, then it is determined that the sound pressure present in the initial audio information is within the preset audio signal sound pressure range; otherwise, if the sound pressure of the initial audio information does not meet any of the above three conditions, that is, the sound pressure of the initial audio information does not fall into the range of 33.58 dBSPL to 39.58 dBSPL, or the sound pressure of the initial audio information does not fall into the range of 33.58 dBSPL to 39.58 dBSPL or the sound pressure of the initial audio information does not fall into the range of 13.3 dBSPL to 16.3 dBSPL, then it is determined that the sound pressure of the initial audio information is not within the preset audio signal sound pressure range.

Similarly, a frequency of the obtained snore complex sound is 160 to 190 Hz, at the moment, 160 to 190 Hz is used as the preset audio signal frequency range, then the frequency of the initial audio information is determined, if the frequency of the initial audio information falls into the range of 160 to 190 Hz, then it is determined that the frequency of the initial audio information is within the preset audio signal frequency range, otherwise, it is determined that the frequency of the initial audio information is not within the preset audio signal frequency range.

It can be understood that, a plurality of preset audio signal frequency ranges and a plurality of preset audio signal sound pressure ranges may be set by relevant personnel according to actual using requirements, which are not limited in the present embodiment.

In some implementations, performing audio information activity detection on the target audio information to obtain the target audio activity information includes:
obtaining an audio signal amplitude and a zero crossing rate corresponding to the initial audio information;
comparing the audio signal amplitude with a preset amplitude threshold to obtain an amplitude threshold comparison result;
comparing the zero crossing rate with a preset zero crossing rate threshold to obtain a zero crossing rate comparison result; and
determining the target audio information as the target audio activity information in a case that the amplitude threshold comparison result indicates that the audio signal amplitude is greater than the amplitude threshold and the zero crossing rate comparison result indicates that the zero crossing rate is less than or equal to the zero crossing rate threshold.

In some implementations, the audio signal amplitude represents the magnitude of a sound corresponding to the initial audio information, the larger the audio signal amplitude of the target audio information, the higher the volume of the sound, otherwise, the smaller the audio signal amplitude of the target audio information, the lower the volume of the sound. The above preset amplitude threshold may be set by relevant personnel according to actual using requirements, the audio signal amplitude and the preset amplitude threshold are compared to obtain the amplitude threshold comparison result, and if the audio signal amplitude is higher than the preset amplitude threshold, it is determined that the audio signal amplitude of the target audio information meets the amplitude threshold, and the target audio information is determined as the target audio activity information; otherwise, if the audio signal amplitude is not higher than the preset amplitude threshold, it is determined that the audio signal amplitude of the target audio information does not meet the amplitude threshold, and the target audio information is not determined as the target audio activity information.

In some implementations, the zero crossing rate (ZCR) is a number of times a sampling value of a sound signal passes through a zero point in each frame of the sound signal, and the preset zero crossing rate threshold may be determined based on a number of times of periodic signals of a sampling number. In some implementations, the periodic signals of the sampling number may be directly used as the preset zero crossing rate threshold. It can be understood that, this example is not used to limit a manner for determining the preset zero crossing rate threshold which is merely determined based on the number of times of the periodic signals of the sampling number, and relevant personnel may flexibly select the manner for determining the preset zero crossing rate threshold. The zero crossing rate is compared with the preset zero crossing rate threshold to obtain the zero crossing rate comparison result, if the zero crossing rate is lower than the preset zero crossing rate threshold, it indicates that the target audio information is a usable normal audio, and at the moment, the target audio information is determined as the target audio activity information; and if the zero crossing rate is higher than the zero crossing rate threshold, it indicates that the target audio information is noise, and at the moment, the target audio information is not determined as the target audio activity information.

In some implementations, taking an example that the periodic signals of the sampling number are directly used as the preset zero crossing rate threshold, after the zero crossing rate of the target audio information is obtained, the zero crossing rate of the target audio information is compared with the periodic signals of the sampling number of the target audio information; if the number of times of the periodic signals of the sampling number is greater than the zero crossing rate, the target audio information is determined as the target audio activity information; otherwise, if the number of times of the periodic signals is less than the zero crossing rate, the target audio information is determined as noise, and the target audio information is not determined as the target audio activity information.

In some implementations, performing audio information activity detection on the target audio information to obtain the target audio activity information includes: obtaining an audio signal amplitude or a zero crossing rate corresponding to the target audio information, the zero crossing rate being a number of times sampling information corresponding to the target audio information crosses a zero point, and the sampling information being obtained after performing many times of sampling on the target audio information; comparing the audio signal amplitude with a preset amplitude threshold to obtain an amplitude threshold comparison result; or comparing the zero crossing rate with a preset zero crossing rate threshold to obtain a zero crossing rate comparison result; and determining the target audio information as the target audio activity information in a case that the amplitude threshold comparison result indicates that the audio signal amplitude is greater than the amplitude threshold or the zero crossing rate comparison result indicates that the zero crossing rate is less than or equal to the zero crossing rate threshold.

In some implementations, the audio signal amplitude corresponding to the initial audio information may be obtained alone, in a case that the amplitude threshold comparison result indicates that the audio signal amplitude is greater than the amplitude threshold, the target audio information is determined as the target audio activity information, otherwise, the target audio information is not determined as the target audio activity information. In some implementations, the zero crossing rate corresponding to the initial audio information may be obtained alone; and in a case that the zero crossing rate comparison result indicates that the zero crossing rate is less than or equal to the zero crossing rate threshold, the target audio information is determined as the target audio activity information, otherwise, the target audio information is not determined as the target audio activity information. In some implementations, it is required to determine the target audio information as the target audio activity information in a case that the amplitude threshold comparison result indicates that the audio signal amplitude is greater than the amplitude threshold, and, the zero crossing rate comparison result indicates that the zero crossing rate is less than or equal to the zero crossing rate threshold.

In some implementations, the sound source positioning parameters include a time difference of the respective sound receiving assemblies receiving audio signals, a spacing distance between the respective sound receiving assemblies, an audio signal propagation velocity and a sampling rate of the target audio information, and performing sound source positioning on the sound producing object corresponding to the target audio activity information according to the sound source positioning parameters corresponding to the target audio activity information to obtain the target position information of the sound producing object includes: performing angle positioning on the sound producing object based on the time difference of the respective sound receiving assemblies receiving the audio signals, the spacing distance between the respective sound receiving assemblies, the audio signal propagation velocity and the sampling rate of the target audio information, to obtain azimuth angle parameters between the sound producing object and the respective sound receiving assemblies; performing distance positioning estimation on the sound producing object based on the audio signal propagation velocity and a time difference of the respective sound receiving assemblies receiving audio signals in front and back cycles, to obtain linear distance parameters between the sound producing object and the sound receiving assemblies; and determining relative position information between the sound producing object and the sound receiving assemblies in a three-dimensional space according to the azimuth angle parameters and the linear distance parameters, and using the relative position information as the target position information.

In some implementations, taking an example that the sound receiving assemblies are arranged on left and right sides respectively, the sound receiving assemblies on the left and right sides receive the audio signals at different time due to a spacing therebetween, there is a time difference of receiving signals between the leftmost sound receiving assembly and the right sound receiving assembly, the azimuth angle parameter of the sound producing object corresponding to the audio signals may be estimated according to the time difference of the two sound receiving assemblies receiving the audio signals, the spacing distance, the audio signal propagation sound velocity and the sampling rate, linear distances between the sound producing object corresponding to the audio signals and the respective sound receiving assemblies may be estimated according to the time difference of the sound receiving assemblies receiving the audio signals in front and back cycles and the audio signal propagation velocity, and after the linear distance parameters and the azimuth angle parameters are obtained, the relative position information between the sound producing object and the sound receiving assemblies in the three-dimensional space is determined based on the linear distance parameters and the azimuth angle parameters, and the relative position information is used as the target position information.

In some implementations, after sound source positioning is performed on the sound producing object corresponding to the target audio activity information to obtain the target position information of the sound producing object, the method further includes: adjusting an image obtaining region corresponding to an infrared image obtaining module according to the target position information to obtain a target image obtaining region, the target image obtaining region including the sound producing object; and executing an infrared image shooting operation on the target image obtaining region through the infrared image obtaining module to obtain infrared image attitude information of the sound producing object, and storing the infrared image attitude information, the infrared image attitude information being used for obtaining a corresponding attitude correction method. That is, according to the target position information of the sound producing object, the image obtaining region of the infrared image obtaining module is adjusted to be the region containing the sound producing object, such that the infrared image obtaining module can shoot the sound producing object to obtain the infrared image attitude information of the sound producing object. It can be understood that, the infrared image obtaining module can execute the infrared image shooting operation on the target image obtaining region under sufficient light to obtain the infrared image attitude information of the sound producing object. Meanwhile, due to the characteristics of infrared rays, the infrared image obtaining module can further execute the infrared image shooting operation on the target image obtaining region in a dark (insufficient light) situation to obtain the infrared image attitude information of the sound producing object, such that this solution can adapt to a scenario where a light change degree is high.

In some implementations, taking an example that the preset audio signal frequency range and the preset audio signal sound pressure range are determined according to the snore complex sound frequency and the decibel-sound pressure level corresponding to snoring, in a case that the audio recognition result indicates that the initial audio information meets the preset audio recognition condition, it indicates that the sound producing object is snoring right now, and after sound source positioning is performed on the snoring sound producing object to obtain the target position information of the snoring sound producing object, the image obtaining region corresponding to the infrared image obtaining module is adjusted to obtain the target image obtaining region; and the infrared image shooting operation is executed on the target image obtaining region through the infrared image obtaining module to obtain the infrared image attitude information of the snoring sound producing object, and the infrared image attitude information is stored.

In some implementations, the infrared image attitude information includes attitude information to be corrected, the attitude information to be corrected is attitude information corresponding to the time when the sound producing object produces the target audio information, and after the infrared image attitude information is stored, the method further includes: searching for an attitude correction method corresponding to the attitude information to be corrected; and showing attitude reminding information corresponding to the attitude correction method to the sound producing object.

In some implementations, taking an example that the sound producing object is the snoring sound producing object, different attitudes will make a snoring degree of the sound producing object different, therefore, the corresponding attitude correction method may be searched for based on the obtained attitude information to be corrected, and the attitude reminding information corresponding to the attitude correction method is shown to the sound producing object, such that the sound producing object can perform attitude correction according to the attitude correction method.

According to the sound source determining method provided by the present embodiment, the sound source determining method includes: obtaining the initial audio information collected in real time; performing audio recognition processing on the initial audio information to obtain the audio recognition result; using the initial audio information corresponding to the audio recognition result as the target audio information in a case that the audio recognition result indicates that the initial audio information meets the preset audio recognition condition; performing audio information activity detection on the target audio information to obtain the target audio activity information; and performing sound source positioning on the sound producing object corresponding to the target audio activity information according to the sound source positioning parameters corresponding to the target audio activity information to obtain the target position information of the sound producing object. In a case that the audio recognition result indicates that the initial audio information meets the preset audio recognition condition, it indicates that the sound producing object corresponding to the initial audio information is a particular sound producing object, after audio information activity detection is performed according to the target audio information to obtain the target audio activity information, sound source positioning is performed on the sound producing object directly according to the target audio information, the target position information of the particular sound producing object is obtained quickly and accurately, and the problem that the particular sound producing object cannot be positioned in the related art is avoided.

### Embodiment 2

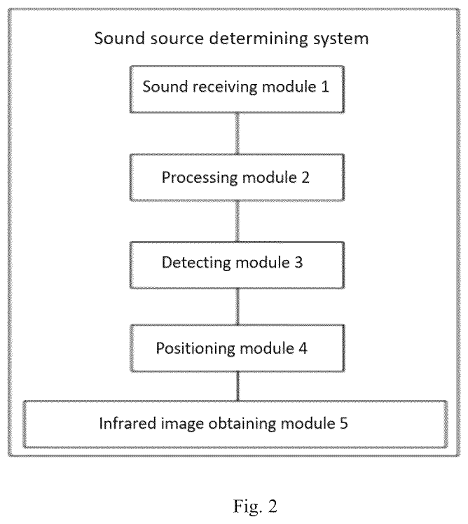

Based on the same technical concept, the present embodiment further provides a sound source determining system, and as shown in FIG. 2, the system includes:
a sound receiving module 1, configured to obtain initial audio information collected in real time;
a processing module 2, configured to perform audio recognition processing on the initial audio information to obtain an audio recognition result; and use the initial audio information corresponding to the audio recognition result as target audio information in a case that the audio recognition result indicates that the initial audio information meets a preset audio recognition condition;
a detecting module 3, configured to perform audio information activity detection on the target audio information to obtain target audio activity information; and
a positioning module 4, configured to perform sound source positioning on a sound producing object corresponding to the target audio activity information according to sound source positioning parameters corresponding to the target audio activity information to obtain target position information of the sound producing object.

The sound source determining system further includes an infrared image obtaining module 5, and the infrared image obtaining module 5 is configured to adjust an image obtaining region corresponding to the infrared image obtaining module 5 according to the target position information to obtain a target image obtaining region, the target image obtaining region including the sound producing object; and an infrared image shooting operation is executed on the target image obtaining region through the infrared image obtaining module 5 to obtain infrared image attitude information of the sound producing object, and the infrared image attitude information is used for obtaining a corresponding attitude correction method.

Obtaining the initial audio information collected in real time includes: arranging sound receiving assemblies in at least two different azimuths, and performing audio information collection in real time based on the sound receiving assemblies in the at least two different azimuths to obtain the initial audio information.

In some implementations, the preset audio recognition condition includes a preset audio signal frequency range and a preset audio signal sound pressure range, and performing audio recognition processing on the initial audio information to obtain the audio recognition result includes: performing frequency feature recognition or sound pressure feature recognition on the initial audio information to obtain the audio recognition result; and using the initial audio information corresponding to the audio recognition result as the target audio information in a case that the audio recognition result indicates that the initial audio information meets the preset audio recognition condition includes: using the initial audio information corresponding to the audio recognition result as the target audio information if the audio recognition result indicates that a frequency of the initial audio information meets the preset audio signal frequency range and a sound pressure of the initial audio information meets the preset audio signal sound pressure range.

In some implementations, performing audio information activity detection on the target audio information to obtain the target audio activity information includes: obtaining an audio signal amplitude and a zero crossing rate corresponding to the target audio information, the zero crossing rate being a number of times sampling information corresponding to the target audio information crosses a zero point, and the sampling information being obtained after performing many times of sampling on the target audio information; comparing the audio signal amplitude with a preset amplitude threshold to obtain an amplitude threshold comparison result; comparing the zero crossing rate with a preset zero crossing rate threshold to obtain a zero crossing rate comparison result; and determining the target audio information as the target audio activity information in a case that the amplitude threshold comparison result indicates that the audio signal amplitude is greater than the amplitude threshold and the zero crossing rate comparison result indicates that the zero crossing rate is less than or equal to the zero crossing rate threshold.

In some implementations, performing audio information activity detection on the target audio information to obtain the target audio activity information includes: obtaining an audio signal amplitude or a zero crossing rate corresponding to the target audio information, the zero crossing rate being a number of times sampling information corresponding to the target audio information crosses a zero point, and the sampling information being obtained after performing many times of sampling on the target audio information; comparing the audio signal amplitude with a preset amplitude threshold to obtain an amplitude threshold comparison result; or comparing the zero crossing rate with a preset zero crossing rate threshold to obtain a zero crossing rate comparison result; and determining the target audio information as the target audio activity information in a case that the amplitude threshold comparison result indicates that the audio signal amplitude is greater than the amplitude threshold or the zero crossing rate comparison result indicates that the zero crossing rate is less than or equal to the zero crossing rate threshold.

In some implementations, the sound source positioning parameters include a time difference of the respective sound receiving assemblies receiving audio signals, a spacing distance between the respective sound receiving assemblies, an audio signal propagation velocity and a sampling rate of the target audio information, and performing sound source positioning on the sound producing object corresponding to the target audio activity information according to the sound source positioning parameters corresponding to the target audio activity information to obtain the target position information of the sound producing object includes: performing angle positioning on the sound producing object based on the time difference of the respective sound receiving assemblies receiving the audio signals, the spacing distance between the respective sound receiving assemblies, the audio signal propagation velocity and the sampling rate of the target audio information, to obtain azimuth angle parameters between the sound producing object and the respective sound receiving assemblies; performing distance positioning estimation on the sound producing object based on the audio signal propagation velocity and a time difference of the respective sound receiving assemblies receiving audio signals in front and back cycles, to obtain linear distance parameters between the sound producing object and the sound receiving assemblies; and determining relative position information between the sound producing object and the sound receiving assemblies in a three-dimensional space according to the azimuth angle parameters and the linear distance parameters, and using the relative position information as the target position information.

In some implementations, after sound source positioning is performed on the sound producing object corresponding to the target audio activity information to obtain the target position information of the sound producing object, the method further includes: adjusting the image obtaining region corresponding to the infrared image obtaining module according to the target position information to obtain the target image obtaining region, the target image obtaining region including the sound producing object; and executing the infrared image shooting operation on the target image obtaining region through the infrared image obtaining module to obtain the infrared image attitude information of the sound producing object, the infrared image attitude information being used for obtaining the corresponding attitude correction method.

It can be understood that, the sound source determining system includes: a processor, an audio processor, a filter, an audio amplifier, an image sensor, a LENS module, an infrared light emitting diode module (IR LED module), an ambient light sensor (ALS), an infrared light filter (IR CUT), a memory, a precise capacitive microphone and other devices, and the above devices jointly constitute the sound receiving module 1, the processing module 2, the detecting module 3, the positioning module 4 and the infrared image obtaining module 5 above.

It is to be understood that, the combination of the modules of the sound source determining system provided by the present embodiment can implement the steps of the above sound source determining method to achieve the technical effects the same as those of the steps of the sound source determining method, which are not repeated here.

### Embodiment 3

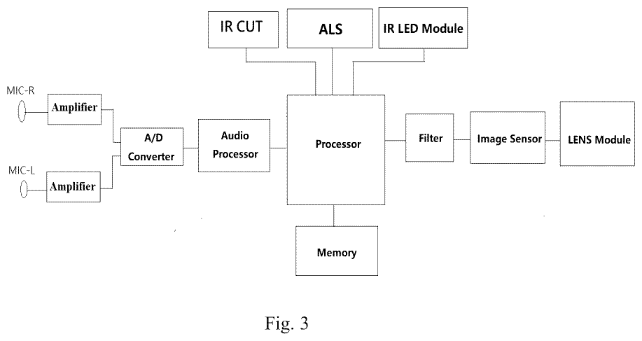

The present embodiment provides a more specific example for description. The present embodiment provides a sound source determining method, the method is applied to a sound source determining system, as shown in FIG. 3, the sound source determining system includes: a processor, an audio processor, a filter, an audio amplifier, an image sensor, a LENS module, an infrared light emitting diode module (IR LED module), an ambient light sensor (ALS), an infrared light filter (IR CUT), a memory, a precise capacitive microphone and other devices, and the above devices jointly constitute a sound receiving module, a processing module, a detecting module, a positioning module and a shooting module in the sound source determining system.

In some implementations, the LENS module is connected with the image sensor, the image sensor is connected with the filter, the filter is connected with the processor, the processor is connected with the memory, and the processor is further connected with the ALS, the IR CUT and the IR LED module.

In some implementations, the ambient light sensor (ALS) is a sensor which can sense an ambient light intensity. The infrared light filter (IR-CUT) has a function of blocking the entrance of infrared rays, only allowing visible rays to enter a camera or a video camera. In this way, the image resolution may be guaranteed, and image distortion or aberration caused by the interference of infrared rays is avoided. In the daytime, the infrared light filter will allow visible rays to enter the camera or the video camera to shoot colorful images. At night or in a dark environment, the infrared light filter will be switched to an infrared ray transmission mode automatically, so as to shoot a black-and-white picture, and thus the shot image has the best effect. The infrared light emitting diode unit (IR-LED) is an electronic element that emits infrared light waves, and is mainly used in applications such as infrared communication, infrared remote controls and infrared sensors. It can convert electric energy into infrared energy, emitted infrared rays can propagate in air and be received by a photosensitive element at a receiving end, and thus the functions such as communication, remote control and detection are achieved. The IR-LED has the characteristics such as long emitting distance, high anti-interference capacity, low power consumption and small size, and is widely applied in the fields such as smart home, industrial automation and security and protection monitoring. In some implementations, the IR-LED includes an infrared transmitter light emitting diode (IR transmitter LED) and an infrared receiver light emitting diode (IR receiver LED), the IR transmitter LED is used for emitting infrared rays, and the IR receiver LED is used for recognizing infrared ray information and transmitting the recognized infrared ray information to the image sensor.

The LENS module is one of lens modules and may be used in the fields such as optical imaging, optical measurement and optical communication. It is typically composed of a set of lenses, a light filter, a diaphragm and other optical elements, and by adjusting the relative positions and angles of these elements, a propagation direction, a focusing effect, light intensity distribution and other optical parameters of light rays may be changed, thereby achieving processing and control of optical signals. The LENS module has the advantages of being simple in structure, easy to adjust, reusable and the like, and is widely applied to various optical systems.

In some implementations, the system includes the image sensor and the filter to be used for collecting light signals in a region where an object to be monitored is located; and meanwhile, it is further equipped with the IR LED module, the ALS and the IR-CUT to assist the image sensor in obtaining the light signals in a case of insufficient illuminance. The IR LED module, the ALS and the IR-CUT will sense an existing ambient light situation and send this information to the processor. The processor will control the work of the IR LED module, the ALS and the IR-CUT according to the information and thus adjust the image sensor, so that the image sensor can normally work in an environment with insufficient illuminance and then collect the light signals.

In some implementations, the two microphones are connected with corresponding audio amplifiers respectively, the audio amplifiers are connected with an A/D converter, the A/D converter is connected with the audio processor, the audio processor is connected with the processor, and the two microphones are connected with the corresponding audio amplifiers and the A/D converter and jointly function with the audio processor to collect audio information.

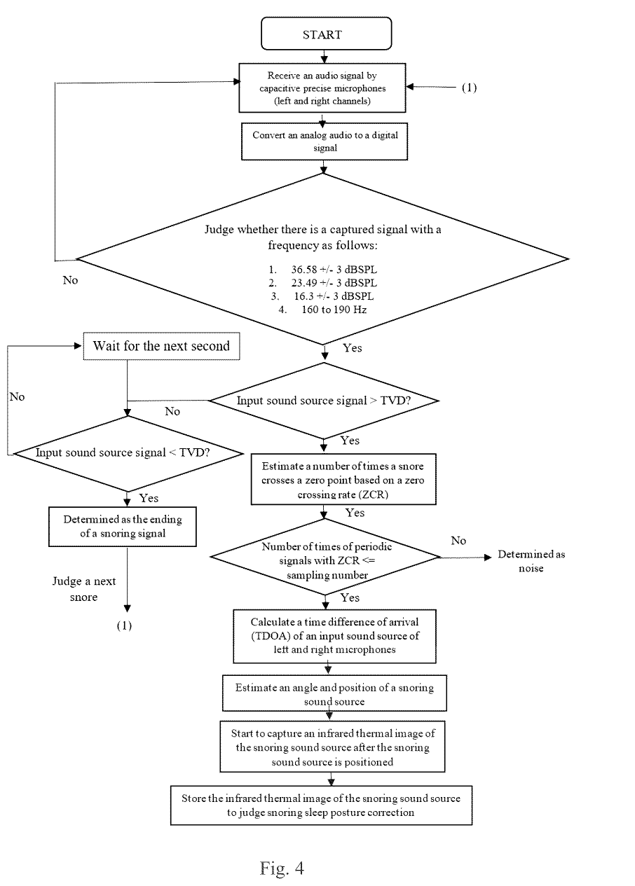

Taking an example that sound source positioning is performed on the snoring sound producing object through the above sound source determining method, as shown in FIG. 4, FIG. 4 shows a schematic flow diagram of the sound source determining method provided by the present embodiment, first, the two capacitive high-precision microphones on the left and right sides of the sound source determining system respectively are arranged on the left and right sides of a sound receiving region respectively and used for receiving decibel-sound pressure levels (dBSPL) during snoring of each region of the throat at the time of snoring within the range of the region so as to obtain audio information, and then the A/D converter converts the audio information to a digital signal according to an analog electric signal. As for the decibel-sound pressure levels during snoring of each region of the throat at the time of snoring: (1) a snore complex sound frequency is: 160 to 190 Hz; (2) an average decibel-sound pressure level of a snore at the soft palate is 36.58 dBSPL; (3) an average decibel-sound pressure level of a snore at the epiglottis is 23.49 dBSPL; and (4) an average decibel-sound pressure level of a snore at the root of tongue is 16.3 dBSPL. After it is confirmed that the decibel-sound pressure level is indeed a snore, a positioning procedure for the snoring sound producing object is started, first, a procedure of sampling a received audio signal and processing the analog signal to convert it to the digital signal is carried out to confirm whether an audio signal amplitude is greater than a threshold value, which is threshold value detection (TVD), the threshold value may be found out by many times of adjustment via a training manner, and the threshold value may be dynamically adjusted according to mass data statistics. Afterwards, a zero crossing rate (ZCR) is judged, the number of times a sampled signal crosses a zero point may be estimated through calculation of the ZCR, the number of times of periodic signals with a ZCR<=a sampling number represents that sampled data are not noise, and the number of times of periodic signals with a ZCR>a sampling number represents that the sampled data are noise, this is because values of left and right channels may cause significant differences in the waveform of the left and right channels, resulting in echo noise. When a captured sound source signal is less than the TVD, it represents the ending of a snoring signal. The two capacitive high-precision microphones of the left and right channels receive a snoring sound source signal at different time due to a spacing therebetween, a time difference of the sound between the leftmost microphone and the right microphone is the maximum, an azimuth angle of the snore may be estimated according to the time difference of the two MICs receiving the audio signal, a distance between the two MICs, a sound velocity and a sampling rate, a linear distance between the snore and the system may be estimated according to the time difference of receiving the snore audio early and later and the sound velocity, and thus the azimuth angle and the relative linear distance between the snore and the snoring system may be estimated.

Capturing of an infrared thermal image is started after the snore sound source position is determined, the infrared thermal image of a sleeping posture of a snorer is captured and then stored, thus it may be not necessary to find out who is the snorer among a plurality of sleepers within the region range and in dark, and the captured infrared thermal image of the sleeping posture of the snorer may be used as a correction reference and a reference for finding out non-invasive snoring treatment.

### Embodiment 4

The present embodiment further provides an electronic device, including one or more processors; and a storage apparatus, wherein the storage apparatus is configured to store one or more computer programs, and the one or more computer programs, when executed by the one or more processors, cause the electronic device to implement the sound source determining method of the present disclosure.

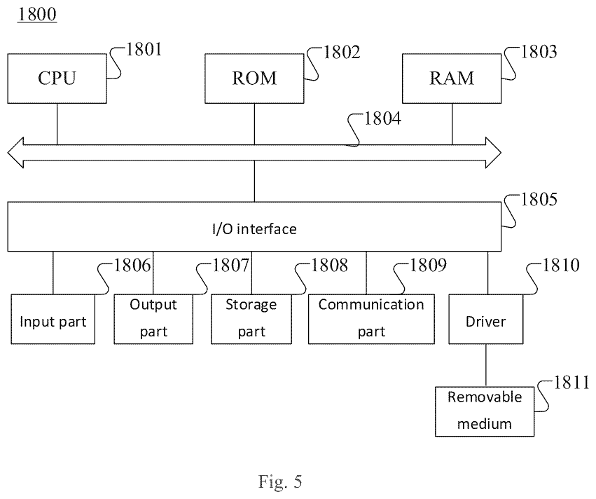

FIG. 5 shows a schematic structural diagram of a computer system of an electronic device suitable for implementing one embodiment of the present disclosure.

It should be noted that, the computer system 1800 of the electronic device shown in FIG. 5 is only an example, and should not limit the function and scope of use of the present embodiment of the present disclosure.

As shown in FIG. 5, the computer system 1800 includes a central processing unit (CPU) 1801, which may execute various appropriate actions and processing, such as executing the method in the above embodiment, according to programs stored in a read-only memory (ROM) 1802 or programs loaded from a storage part 1808 onto a random access memory (RAM) 1803. Various programs and data required for the operation of the system are further stored in the RAM 1803. The CPU 1801, the ROM 1802, and the RAM 1803 are connected to each other through a bus 1804. An input/output (I/O) interface 1805 is also connected to the bus 1804.

In some implementations, the following components are connected to the I/O interface 1805: an input part 1806 including a keyboard, a mouse and the like; an output part 1807 including a cathode ray tube (CRT), a liquid crystal display (LCD), a loudspeaker and the like; a storage part 1808 including a hard disk and the like; and a communication part 1809 including network interface cards such as a local area network (LAN) card and a modem. The communication part 1809 performs communication processing via a network such as the Internet. A driver 1810 is further connected to the I/O interface 1805 as required. A removable medium 1811, such as a magnetic disk, an optical disk, a magneto-optical disk and a semiconductor memory, are installed on the driver 1810 as required, so that computer programs read from it can be installed into the storage part 1808 as required.

In some implementations, according to the embodiments of the present disclosure, the process described with reference to the flow diagrams above may be implemented as a computer program. For example, an embodiment of the present disclosure includes a computer program product, the computer program product includes a computer program carried on a computer-readable medium, and the computer program includes a computer program used for executing the method shown in the flow diagrams. In such embodiment, the computer program may be downloaded from the network through the communication part 1809 and installed, and/or installed from the removable medium 1811. The computer program, when executed by the CPU 1801, executes various functions defined in the system of the present disclosure.

It should be noted that, the computer-readable medium shown in the embodiment of the present disclosure may be a computer-readable signal medium or a computer-readable storage medium or any combination of the two. The computer-readable storage medium may be, for example, an electrical, magnetic, optical, electromagnetic, infrared, or semiconductor system, apparatus, or device, or any combination of the above. More specific examples of the computer-readable storage medium may include but are not limited to: electrical connections with one or more wires, a portable computer magnetic disk, a hard disk, a random access memory (RAM), a read-only memory (ROM), an erasable programmable read only memory, a flash, an optical fiber, a portable compact disc read-only memory (CD-ROM), an optical storage device, a magnetic storage device or any suitable combination of the above. In the present disclosure, the computer-readable signal medium may include a data signal propagated in a baseband or as part of a carrier wave, in which a computer-readable computer program is carried. This propagated data signal may take a variety of forms, including, but not limited to, electromagnetic signals, optical signals, or any suitable combination of the above. The computer-readable signal medium may also be any computer-readable medium other than the computer-readable storage medium, and the computer-readable medium may send, propagate, or transmit a program for use by or use in combination with an instruction execution system, apparatus, or device. The computer program contained on the computer-readable medium may be transmitted by any suitable medium, including but not limited to: wireless, wired, etc., or any suitable combination of the above.

The flow diagrams and block diagrams in the accompanying drawings illustrate system architectures, functions and operations that may be implemented by an apparatus, a method, and a computer program product according to various embodiments of the present disclosure. Each box in the flow diagrams or the block diagrams may represent a module, a program segment, or part of a code. The module, the program segment, or the part of the code above includes one or more executable instructions used for implementing designated logic functions. It is also to be noted that, in some alternative implementations, functions marked in the blocks may also occur in an order different from that marked in the accompanying drawings. For instance, two successively represented blocks may actually be executed in parallel substantially, and sometimes they may also be executed in an inverse order, which depends on involved functions. It is also to be noted that, each box in the block diagrams or the flow diagrams and a combination of boxes in the block diagrams or the flow diagrams may be implemented by using a dedicated hardware-based system configured to perform a specified function or operation, or may be implemented by using a combination of dedicated hardware and a computer program.

The involved units or modules described in the embodiments of the present disclosure may be implemented in a software form, or a hardware form, and the described units or modules may also be arranged in a processor. The names of these units or modules do not constitute a limitation on the units or modules themselves in a certain situation.

Another aspect of the present disclosure further provides a computer-readable storage medium on which a computer program is stored, and the computer program, when executed by a processor, implements the sound source determining method of the present disclosure. The computer-readable storage medium may be included in the electronic device described in the above embodiment, or may exist separately without being assembled into the electronic device.

Another aspect of the present disclosure further provides a computer program product, including a computer program, and the computer program is stored in a computer-readable storage medium. A processor of an electronic device reads the computer program from the computer-readable storage medium, and the processor executes the computer program to cause the electronic device to execute the sound source determining method as described previously in the various embodiments above.

It should be noted that, although a plurality of modules or units of the device used for action execution are mentioned in the detailed description above, this division is not mandatory. In fact, according to the implementations of the present disclosure, the features and functions of two or more modules or units described above may be concretized within one module or unit. On the contrary, the features and functions of a module or unit described above may be further divided to be concretized by a plurality of modules or units.

Other implementations of the present disclosure will be apparent to those skilled in the art from consideration of the specification and practice of the implementations disclosed herein. The present disclosure is intended to cover any variations, uses, or adaptations of the present disclosure, and these variations, uses, or adaptations follow the general principles of the present disclosure and include such departures from the present disclosure as come within known or customary practice in the art.

The above content is merely certain exemplary embodiments of the present disclosure and is not intended to limit the implementations of the present disclosure. Those ordinarily skilled in the art can quite conveniently make corresponding changes or modifications according to the main ideas and spirit of the present disclosure. Therefore, the scope of protection of the present disclosure shall be based on the scope of protection required by the claims.

What is claimed is:

1. A sound source determining method, comprising:
obtaining initial audio information collected in real time;
performing audio recognition processing on the initial audio information to obtain an audio recognition result;
using the initial audio information corresponding to the audio recognition result as target audio information in a case that the audio recognition result indicates that the initial audio information meets a preset audio recognition condition;
performing audio information activity detection on the target audio information to obtain target audio activity information; and
performing sound source positioning on a sound producing object corresponding to the target audio activity information according to sound source positioning parameters corresponding to the target audio activity information to obtain target position information of the sound producing object.

2. The method of claim 1, wherein obtaining the initial audio information collected in real time comprises:
arranging sound receiving assemblies in at least two different azimuths, and performing audio information collection in real time based on the sound receiving assemblies in the at least two different azimuths to obtain the initial audio information.

3. The method of claim 2, wherein the sound receiving assemblies are microphones and obtaining the initial audio information collected in real time comprises arranging the microphones in at least two different azimuths of one region respectively and performing audio information collection in real time through the at least two microphones synchronously to obtain the initial audio information, wherein the at least two microphones work synchronously when performing audio information collection.

4. The method of claim 1, wherein the preset audio recognition condition comprises a preset audio signal frequency range and a preset audio signal sound pressure range, and performing audio recognition processing on the initial audio information to obtain the audio recognition result comprises:
performing frequency feature recognition or sound pressure feature recognition on the initial audio information to obtain the audio recognition result; and
using the initial audio information corresponding to the audio recognition result as the target audio information in a case that the audio recognition result indicates that the initial audio information meets the preset audio recognition condition comprises:
using the initial audio information corresponding to the audio recognition result as the target audio information if the audio recognition result indicates that a frequency of the initial audio information meets the preset audio signal frequency range and a sound pressure of the initial audio information meets the preset audio signal sound pressure range.

5. The method of claim 1, wherein performing audio recognition processing on the initial audio information to obtain an audio recognition result comprises:
obtaining the audio signal frequency or the audio signal sound pressure of the initial audio information, then matching the frequency of the initial audio information with the preset audio signal frequency range and matching the sound pressure of the initial audio information with the preset audio signal sound pressure range; and
determining that the initial audio information corresponding to the audio recognition result is used as the target audio information in a case that the audio recognition result indicates that the initial audio information meets the preset audio recognition condition, wherein the frequency of the initial audio information is within the preset audio signal frequency range and the sound pressure of the initial audio information is within the preset audio signal sound pressure range.

6. The method of claim 1, wherein performing audio recognition processing on the initial audio information to obtain an audio recognition result comprises:
obtaining the audio signal frequency or the audio signal sound pressure of the initial audio information, then matching the frequency of the initial audio information with the preset audio signal frequency range and matching the sound pressure of the initial audio information with the preset audio signal sound pressure range; and
determining that the initial audio information corresponding to the audio recognition result is not used as the target audio information in a case that the audio recognition result indicates that the initial audio information does not meet the preset audio recognition condition, wherein the frequency of the initial audio information is not within the preset audio signal frequency range or the sound pressure of the initial audio information is not within the preset audio signal sound pressure range.

7. The method of claim 1, wherein performing audio information activity detection on the target audio information to obtain the target audio activity information comprises:
obtaining an audio signal amplitude and a zero crossing rate corresponding to the target audio information, the zero crossing rate being a number of times sampling information corresponding to the target audio information crosses a zero point, and the sampling information being obtained after performing many times of sampling on the target audio information;
comparing the audio signal amplitude with a preset amplitude threshold to obtain an amplitude threshold comparison result;
comparing the zero crossing rate with a preset zero crossing rate threshold to obtain a zero crossing rate comparison result; and
determining the target audio information as the target audio activity information in a case that the amplitude threshold comparison result indicates that the audio signal amplitude is greater than the amplitude threshold and the zero crossing rate comparison result indicates that the zero crossing rate is less than or equal to the zero crossing rate threshold.

8. The method of claim 7, wherein the audio signal amplitude represents the magnitude of a sound corresponding to the initial audio information, the larger the audio signal amplitude of the target audio information, the higher the volume of the sound, otherwise, the smaller the audio signal amplitude of the target audio information, the lower the volume of the sound.

9. The method of claim 1, wherein performing audio information activity detection on the target audio information to obtain target audio activity information
obtaining the zero crossing rate of the target audio information, and using the periodic signals of the sampling number as the preset zero crossing rate threshold,
comparing the zero crossing rate of the target audio information with the periodic signals of the sampling number of the target audio information; if the number of times of the periodic signals of the sampling number is greater than the zero crossing rate, the target audio information is determined as the target audio activity information; if the number of times of the periodic signals is less than the zero crossing rate, the target audio information is determined as noise, and the target audio information is not determined as the target audio activity information.

10. The method of claim 1, wherein performing audio information activity detection on the target audio information to obtain the target audio activity information comprises:
obtaining an audio signal amplitude corresponding to the target audio information;
comparing the audio signal amplitude with a preset amplitude threshold to obtain an amplitude threshold comparison result; and
determining the target audio information as the target audio activity information, wherein the amplitude threshold comparison result indicates that the audio signal amplitude is greater than the amplitude threshold.

11. The method of claim 1, wherein performing audio information activity detection on the target audio information to obtain the target audio activity information comprises:
obtaining a zero crossing rate corresponding to the target audio information, the zero crossing rate being a number of times sampling information corresponding to the target audio information crosses a zero point, and the sampling information being obtained after performing many times of sampling on the target audio information;
comparing the zero crossing rate with a preset zero crossing rate threshold to obtain a zero crossing rate comparison result; and
determining the target audio information as the target audio activity information, wherein the zero crossing rate comparison result indicates that the zero crossing rate is less than or equal to the zero crossing rate threshold.

12. The method of claim 2, wherein the sound source positioning parameters comprise a time difference of the respective sound receiving assemblies receiving audio signals, a spacing distance between the respective sound receiving assemblies, an audio signal propagation velocity and a sampling rate of the target audio information, and performing sound source positioning on the sound producing object corresponding to the target audio activity information according to the sound source positioning parameters corresponding to the target audio activity information to obtain the target position information of the sound producing object comprises:
performing angle positioning on the sound producing object based on the time difference of the respective sound receiving assemblies receiving the audio signals, the spacing distance between the respective sound receiving assemblies, the audio signal propagation velocity and the sampling rate of the target audio information, to obtain azimuth angle parameters between the sound producing object and the respective sound receiving assemblies;
performing distance positioning estimation on the sound producing object based on the audio signal propagation velocity and a time difference of the respective sound receiving assemblies receiving audio signals in front and back cycles, to obtain linear distance parameters between the sound producing object and the sound receiving assemblies; and
determining relative position information between the sound producing object and the sound receiving assemblies in a three-dimensional space according to the azimuth angle parameters and the linear distance parameters, and using the relative position information as the target position information.

13. The method of claim 12, wherein the sound receiving assemblies are arranged on left and right sides, the sound receiving assemblies on the left and right sides receive the audio signals at different time due to a spacing therebetween, there is a time difference of receiving signals between a leftmost sound receiving assembly and a right sound receiving assembly, and performing sound source positioning on the sound producing object corresponding to the target audio activity information according to the sound source positioning parameters corresponding to the target audio activity information to obtain the target position information of the sound producing object
estimating the azimuth angle parameter of the sound producing object corresponding to the audio signals according to the time difference of the two sound receiving assemblies receiving the audio signals, the spacing distance, the audio signal propagation sound velocity and the sampling rate, linear distances between the sound producing object corresponding to the audio signals;
estimating the respective sound receiving assemblies according to the time difference of the sound receiving assemblies receiving the audio signals in front and back cycles and the audio signal propagation velocity;
determining the relative position information between the sound producing object and the sound receiving assemblies in the three-dimensional space based on the linear distance parameters and the azimuth angle parameters, after the linear distance parameters and the azimuth angle parameters are obtained; and
using the relative position information as the target position information.

14. The method of claim 1, wherein after performing sound source positioning on the sound producing object corresponding to the target audio activity information to obtain the target position information of the sound producing object, the method further comprises:
adjusting an image obtaining region corresponding to an infrared image obtaining module according to the target position information to obtain a target image obtaining region, the target image obtaining region comprising the sound producing object; and
executing an infrared image shooting operation on the target image obtaining region through the infrared image obtaining module to obtain infrared image attitude information of the sound producing object, and storing the infrared image attitude information, the infrared image attitude information being used for obtaining a corresponding attitude correction method.

15. The method of claim 14, further comprising:
adjusting the image obtaining region of the infrared image obtaining module to be the region containing the sound producing object, according to the target position information of the sound producing object, such that the infrared image obtaining module can shoot the sound producing object to obtain the infrared image attitude information of the sound producing object.

16. The method of claim 14, wherein the audio recognition result indicates that the initial audio information meets the preset audio recognition condition, it indicates that the sound producing object is snoring right now, and after sound source positioning is performed on the snoring sound producing object to obtain the target position information of the snoring sound producing object, the image obtaining region corresponding to the infrared image obtaining module is adjusted to obtain the target image obtaining region; and the infrared image shooting operation is executed on the target image obtaining region through the infrared image obtaining module to obtain the infrared image attitude information of the snoring sound producing object, and the infrared image attitude information is stored.

17. The method of claim 14, wherein the infrared image attitude information includes attitude information to be corrected, the attitude information to be corrected is attitude information corresponding to the time when the sound producing object produces the target audio information, and after the infrared image attitude information is stored, the method further includes: searching for an attitude correction method corresponding to the attitude information to be corrected; and showing attitude reminding information corresponding to the attitude correction method to the sound producing object.

18. A sound source determining system, comprising:
a sound receiving module, configured to obtain initial audio information collected in real time;
a processing module, configured to perform audio recognition processing on the initial audio information to obtain an audio recognition result; and use the initial audio information corresponding to the audio recognition result as target audio information in a case that the audio recognition result indicates that the initial audio information meets a preset audio recognition condition;
a detecting module, configured to perform audio information activity detection on the target audio information to obtain target audio activity information; and
a positioning module, configured to perform sound source positioning on a sound producing object corresponding to the target audio activity information according to sound source positioning parameters corresponding to the target audio activity information to obtain target position information of the sound producing object.

19. An electronic device, comprising:
one or more processors; and
a storage apparatus, configured to store one or more programs, wherein the one or more programs, when executed by the one or more processors, cause the electronic device to execute the sound source determining method of claim 1.

20. A computer-readable storage medium, storing a computer program thereon, wherein the computer program, when executed by a processor of an electronic device, causes the electronic device to execute the sound source determining method of claim 1.
